(19) United States
(12) Patent Application Publication
(10) Pub. No.: US 2025/0325636 A1
(43) Pub. Date: Oct. 23, 2025

(54) USE OF ENDOTHELIN RECEPTOR TYPE B AGONISTS FOR THE TREATMENT OF AORTIC VALVE STENOSIS

(71) Applicants: INSTITUT NATIONAL DE LA SANTÉ ET DE LA RECHERCHE MÉDICALE, Paris (FR); UNIVERSITÉ DE ROUEN NORMANDIE, Mont-Saint-Aignan (FR); CENTRE HOSPITALIER UNIVERSITAIRE DE ROUEN, Rouen (FR)

(72) Inventors: Jérémy BELLIEN, Rouen cedex (FR); Eric DURAND, Rouen cedex (FR); Vincent RICHARD, Rouen cedex (FR)

(21) Appl. No.: 18/870,937

(22) PCT Filed: Jun. 8, 2023
(86) PCT No.: PCT/EP2023/065358

§ 371 Date: Dec. 2, 2024

(30) Foreign Application Priority Data
Jun. 9, 2022 (EP) 22305833.0

(51) Int. Cl. A61K 38/22; A61F 2/24

(52) U.S. Cl. CPC A61K 38/2285; A61F 2/2412; A61F 2250/0068

(57) ABSTRACT

Aortic valve stenosis (AS), is the most frequent valvular heart disease in Europe and affects more than 1 in 4 people over 65 years old. AS progression from fibrotic thickening to valvular leaflets calcification leads to heart failure development and eventually to death within 2 to 5 years after symptoms occurrence. The inventors now show that endothelin receptor type B (ETB) activation with an agonist decreased the calcium content of VIC. Therefore, the present invention relates to the use of endothelin receptor type B (ETB) agonists for the treatment of aortic valve stenosis.

### FIELD OF THE INVENTION

The present invention is in the field of medicine and in particular in cardiology.

### BACKGROUND OF THE INVENTION

Aortic valve calcification is a condition in which calcium deposits form on the aortic valve in the heart. These deposits can cause narrowing at the opening of the aortic valve. This narrowing can become severe enough to reduce blood flow through the aortic valve—a condition called aortic valve stenosis.

Aortic valve stenosis (AS), is the most frequent valvular heart disease in Europe and affects more than 12.4% of population over 75 years old 3.5% of them presenting a severe form. AS progression from fibrotic thickening to valvular leaflets calcification leads to heart failure development and eventually to death within 2 to 5 years after symptoms occurrence. Its prevalence increases due to rapid aging of population and will become a public health issue within the next years. Up to date there are no pharmacological treatments for this pathology, AS patients being currently treated only with surgical or transcatheter aortic valve replacement (TAVI). Unfortunately, many patients are not eligible for these procedures due to a wide panel of associated co-morbidities, highlighting the critical and urgent need to develop nonsurgical pharmacological treatments.

### SUMMARY OF THE INVENTION

The present invention is defined by the claims. In particular, the present invention relates to the use of Endothelin receptor type B (ET_{B}) agonists for reversing, preventing, or delaying calcification of aortic valve. The present invention also relates to the use of Endothelin receptor type B (ET_{B}) agonists for the treatment of aortic valve stenosis (AS).

### DETAILED DESCRIPTION OF THE INVENTION

The inventors show that Endothelin receptor type B (ET_{B}) agonists decreased the calcium content of VIC and thus represent an attractive therapeutic target to prevent AS progression.

Accordingly, the first object of the present invention relates to a method of treating Aortic valve Stenosis (AS) in a patient in need comprising administering to the patient a therapeutically effective amount of an endothelin receptor type B (ET_{B}) agonist.

Accordingly, the second object of the present invention relates to a method of reversing, preventing, or delaying calcification of aortic valve in a patient in need thereof comprising administering to the patient a therapeutically effective amount of an endothelin receptor type B (ET_{B}) agonist.

As used herein, the terms “subject” or “patient” refer to a mammal, such as a rodent, a feline, a canine, and a primate. Particularly, the subject according to the invention is a human. Particularly, the subject according to the invention is an adult. Particularly, the subject according to the invention is an elder.

As used herein, the term “valve” may refer to the valve that prevents the backflow of blood during the rhythmic contractions. There are four main heart valves. In particular, the aortic valve separates the left ventricle and aorta. The term “aortic valve” includes a diseased aortic valve or a bioprosthetic valve.

As used herein, the term “valve frame” or “prosthetic valve frame” or “valve-in-valve” refers to a three-dimensional structural component, usually tubular, cylindrical, or oval or ring-shaped, and that is seated within a native valve annulus and is used as a mounting element for a commercially available valve.

As used herein, the term “frame” refers to a flexible structure that is mounted within the lumen of a ring or conduit of native tissue, and that surrounds, encircles, or encloses, in part or wholly, an opening or space. As used herein, a “frame” functions to support a leaflet structure or other flow control structure. The valve frame can be a tube, ring, or cylindrical or conical tube, made from a durable, biocompatible structural material such as Nitinol or similar alloy, wherein the valve frame is formed by manufacturing the structural material as a braided wire frame, a laser-cut wire frame, or a wire loop. The valve frame is about 5-60 mm in height, has an outer diameter dimension, R, of 30-80 mm, and an inner diameter dimension of 31-79 mm, accounting for the thickness of the wire material itself. As stated, the valve frame can have a sideprofile of a tubular shape, a ring shape, cylinder shape, conical tube shape, but may also have a side profile of a flat-cone shape, an inverted flat-cone shape (narrower at top, wider at bottom), a concave cylinder (walls bent in), a convex cylinder (walls bulging out), an angular hourglass, a curved, graduated hourglass, a ring or cylinder having a flared top, flared bottom, or both. In one preferred embodiment, the valve frame used for mounting a prosthetic valve deployed in the tricuspid annulus, and may have a complex shape determined by the anatomical structures where the valve frame is being mounted. For example, in the tricuspid annulus, the circumference of the tricuspid valve may be a rounded ellipse, the septal wall is known to be substantially vertical, and the tricuspid is known to enlarge in disease states along the anterior-posterior line. Accordingly, a prosthetic valve may start in a roughly tubular configuration, and be heat-shaped to provide an upper atrial cuff or flange for atrial sealing and a lower trans-annular tubular section having an hourglass cross-section for about 60-80% of the circumference to conform to the native annulus along the posterior and anterior annular segments while remaining substantially vertically flat along 20-40% of the annular circumference to conform to the septal annular segment.

In some embodiment, the valve is a bioprosthetic valve.

As used herein, the term “bioprosthetic valve” is a stented tissue heart valve and may refer to a device used to replace or supplement an aortic valve that is defective, malfunctioning, or missing. Examples of bioprosthetic valve prostheses include, but are not limited to, TS 3fs® Aortic Bioprosthesis, Carpentier-Edwards PERIMOUNT Magna Ease Aortic Heart Valve, Carpentier-Edwards PERIMOUNT Magna Aortic Heart Valve, Carpentier-Edwards PERIMOUNT Magna Mitral Heart Valve, Carpentier-Edwards PERIMOUNT Aortic Heart Valve, Carpentier-Edwards PERIMOUNT Plus Mitral Heart Valve, Carpentier-Edwards PERIMOUNT Theon Aortic Heart Valve, Carpentier-Edwards PERIMOUNT Theon Mitral Replacement System, Carpentier-Edwards Aortic Porcine Bioprosthesis, Carpentier-Edwards Duraflex Low Pressure Porcine Mitral Bioprosthesis, Carpentier-Edwards Duraflex mitral bioprosthesis (porcine), Carpentier-Edwards Mitral Porcine Bioprosthesis, Carpentier-Edwards S.A.V. Aortic Porcine Bioprosthesis, Edwards Prima Plus Stentless Bioprosthesis, Edwards Sapien Transcatheter Heart Valve, Medtronic, Freestyle® Aortic Root Bioprosthesis, Hancock® II Stented Bioprosthesis, Hancock II Ultra® Bioprosthesis, Mosaic® Bioprosthesic, Mosaic Ultra® Bioprosthesis, St. Jude Medical, Biocor®, Biocor™ Supra, Biocor® Pericardia, Biocor™ Stentless, Epic™, Epic Supra™, Toronto Stentless Porcine Valve (SPV®), Toronto SPV II®, Trifecta, Sorin Group, Mitroflow Aortic Pericardial Valve®, Cryolife, Cryolife aortic Valve® Cryolife pulmonic Valve®, Cryolife-O′Brien stentless aortic xenograft Valve®, the Inspiris Resilia aortic valve from Edwards Lifesciences, the Masters HP 15 mm valve from Abbott, the Crown PRT leaflet structure from Livanova/Sorin, the Carbomedics family of valves from Sorin, and all variations thereof. Generally, bioprosthetic valve comprise a tissue valve having one or more cusps and the valve is mounted on a frame or stent, both of which are typically elastical.

In some embodiment, the bioprosthetic valve is a percutaneous aortic valve which is implanted by the method of transcatheter aortic valve implantation (TAVI). Examples of percutaneous aortic valve include but are not limited to Sapien®, Sapien 3®, and Sapien XTR® from Edwards Lifesciences, CoreValve™ Evolut™ R or CoreValve™ Evolut™ PRO from Medtronicor, Lotus Edge valve or ACURATE neo2™ from Boston Scientific.

As used herein, the term “elastical” means that the device is able of flexing, collapsing, expanding, or a combination thereof. The cusps of the valve are generally made from tissue of mammals such as, without limitation, pigs (porcine), cows (bovine), horses, sheep, goats, monkeys, and humans.

As used herein, the term “calcification” has its general meaning in the art and refers to the accumulation of calcium salts in a body tissue (e.g. soft tissue: arteries, cartilage, heart valves . . . ), causing it to harden.

As used herein, the term “valvulopathy” has its general meaning in the art and refers to damage to a heart valve: narrowing of a valve orifice (of a valve) or of an artery, hindering the passage of blood or deficiency. There are 2 types of a dysfunction of the cardiac valves:
the stenosis (narrowing of the valve) wherein the valve does not open sufficiently and the flux of blood is therefore slowed down.
the insufficiency (leakage) wherein the valve is not closing properly and heart has therefore to work more, so that it can function normally.
   These dysfunctions bring along a dilation and a tired heart: being short of breath and the risk of oedemas of the inferior legs, uneasiness, sometimes lost of conscience, palpitations, congestive heart-failure.

In some aspects, the method of the present invention is particularly suitable for preventing stenosis.

As used herein, the term “preventing” intends characterizing a prophylactic method or process that is aimed at delaying or preventing the onset of a disorder or condition to which such term applies.

As used herein, the term “stenosis” refers to the narrowing of the aortic valve that could block or obstruct blood flow from the heart and cause a back-up of flow and pressure in the heart.

As used herein, the term “aortic valve stenosis” or “AS” has its general meaning in the art and refers to formation, growth or deposition of extracellular matrix hydroxyapatite (calcium phosphate) crystal deposits in the aortic valve, including a bioprosthetic valve.

In some embodiments, the method of the present invention is particularly suitable for primary prevention of AS. In particular, the method of the present invention is suitable for the treatment of a subject suffering from calcific aortic valve disease (CAVD).

As used herein, the term “calcific aortic valve disease” or “CAVD” has its general meaning in the art and refers to a slowly progressive disorder with a disease continuum that ranges from mild valve thickening without obstruction of blood flow, termed aortic sclerosis, to severe calcification with impaired leaflet motion, or aortic stenosis. Thus disease progression is generally characterized by a process of thickening of the valve leaflets and the formation of calcium nodules—often including the formation of actual bone—and new blood vessels, which are concentrated near the aortic surface. End-stage disease, e.g., calcific aortic stenosis, is generally characterized pathologically by large nodular calcific masses within the aortic cusps that protrude along the aortic surface into the sinuses of Valsalva, interfering with opening of the cusps. In some embodiments, the method of the present invention is particularly suitable for the treatment of calcific aortic stenosis.

In some embodiments, the method of the present invention is particularly suitable for secondary prevention of calcification. In particular, the method of the present invention is particularly suitable for preventing degeneration of an implanted bioprosthetic valve. Thus in some embodiment, the method of the present invention is particularly suitable for delaying or preventing the calcification of a bioprosthetic valve after valve replacement either surgically or after transcatheter aortic valve implantation (TAVI).

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “endothelins” (ETs) comprises three isoforms, ET-1, ET-2, and ET-3. ETs interact with two distinct G-protein-coupled receptors, ET_{A} and ET_{B}. They are both class A, G-protein-coupled receptors. The initial clue to the existence of two subtypes and the key to classifying the receptors was that ET-1 and ET-2 are equipotent at the ET_{A} subtype whereas ET-3 shows at least 100-fold lower potency and at physiological concentration ET-3 is unlikely to activate this subtype. All three ETs bind to ET_{B} with similar affinity.

As used herein, the term “endothelin 1” (ET-1) has its general meaning in the art and refers to the vasoconstricting peptide endothelin-1 (ET-1) produced primarily in the endothelium and acting on its both receptors ET_{A} and ET_{B}. The endothelin (ET-1) can be from any source, but typically is a mammalian (e.g., human and non-human primate) endothelin, particularly a human endothelin. An exemplary native endothelin-1 amino acid sequence is provided in UniProt database under accession number P05305 and an exemplary native nucleotide sequence encoding for endothelin-1 is provided in GenBank database under accession number NM_001955.

As used herein, the term “endothelin receptor type A” (ET_{A}) has its general meaning in the art and refers to the endothelin receptor type A. ET_{A} can be from any source, but typically is a mammalian (e.g., human and non-human primate) ET_{A}, particularly a human ET_{A}. An exemplary native ET_{A} amino acid sequence is provided in UniProt database under accession number P25101 and an exemplary native nucleotide sequence encoding for ETA is provided in GenBank database under accession number NM_001957.

As used herein, the term “endothelin receptor type B” (ET_{B}) has its general meaning in the art and refers to the endothelin receptor type B. ET_{B} can be from any source, but typically is a mammalian (e.g., human and non-human primate) ETB, particularly a human ET_{B}. An exemplary native ET_{B} amino acid sequence is provided in UniProt database under accession number P24530 and an exemplary native nucleotide sequence encoding for ET_{B} is provided in GenBank database under accession number NM_000115.

As used herein, the term “agonist”, refers to an agent that is capable of specifically binding and activating ET_{B} receptor for initiating a pathway signalling and further biological processes through a receptor to fully activate, as does an activator, or detectably induce or stimulate a response mediated by the receptor.

As used herein, the terms “endothelin type B receptor agonist”, “ET_{B} receptor agonist”, and “ET_{B} agonist” are used interchangeably and refer to a natural or synthetic compound which binds and activates fully or partially ET_{B} for initiating or participating to a pathway signaling and further biological processes. ET_{B} agonistic activity may be assessed by various methods known by the skilled person.

In a particular embodiment, the ET_{B} agonist is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Specific examples of ET_{B} agonists useful in the present invention include, but are not limited to, IRL-1620, ET-3, sarafotoxin 6c, BQ-3020, AIa(1,3,11,15)-Endothelin-1, and mixtures thereof.

As used herein, the term “sarafotoxin 6c” (SFT6C) is a ET_{B} endothelin receptor agonist that induces muscle contractions. SFT6C has a molecular formula of C₁₀₃H₁₄₇N₂₇O₃₇S₅ and the following CAS number: 121695-87-2.

As used herein, the term “Endothelin 3” (ET-3) is an endogenous neuropeptide and potent vasoconstrictor. ET-3 has a molecular formula of C₁₂₁H₁₆₈N₂₆O₃₃S₄ and the following CAS number: 117399-93-6.

As used herein, the term “BQ-3020” also termed (N-Ac-AIa(11,15)-endothelin-1 (6-21)) and N-Aceytyl-[Alal 1,15]-Endothelin 1 fragment 6-21 is a highly potent and selective ET_{B} endothelin receptor agonist. BQ-3020 has a molecular formula of C₉₆H₁₄₀N₂₀O₂₅S; and the following CAS number: 143113-45-5.

As used herein, the term “Ala^{(1,3,11,15)}-Endothelin-1” is a linear endothelin (ET)-1 analogue, and acts as an ET_{B} receptor agonist. Ala^{(1,3,11,15)}-Endothelin-1 has a molecular formula of C₁₀₉H₁₆₃N₂₅O₃₂S and the following CAS Number: 121204-87-3.

In a particular embodiment, the endothelin receptor type B agonist is IRL-1620.

As used herein, the term “IRL-1620” N-Succinyl-[Glu9, Alal U5]-Endothelin 1 fragment 8-21 is a highly selective ET_{B} endothelin also termed receptor agonist. IRL-1620 has a molecular formula of C₈₆H₁₁₇N₁₇O₂₇ and the following CAS number: 142569-99-1

In a particular embodiment, the ET_{B} agonist is a peptide, a peptidomimetic, an antibody or an aptamers.

In a particular embodiment, the ET_{B} agonist is a peptidomimetic. The term “peptidomimetic” refers to a small protein-like chain designed to mimic a peptide.

In a particular embodiment, the ET_{B} agonist is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In some embodiments, the ET_{B} agonist is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab', Fab, F(ab')2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab')2 fragments can be generated by treating the antibody with pepsin. The resulting F(ab')2 fragment can be treated to reduce disulfide bridges to produce Fab' fragments. Papain digestion can lead to the formation of Fab fragments. Fab, Fab' and F(ab')2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the ET_{B} agonist is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the ET_{B} agonist is an intrabody having specificity for ET_{B}. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

As used herein the terms “administering” or “administration” refer to the act of injecting or otherwise physically delivering a substance as it exists outside the body (e.g. a ET_{B} agonist) into the subject, such as by mucosal, intradermal, intravenous, subcutaneous, intramuscular delivery, transdermal delivery (e.g. a portable infusion pump) and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof. In some embodiments, the patient is administered with a pharmaceutical composition comprising the therapeutically effective amount of a ET_{B} agonist as active principle and at least one pharmaceutically acceptable excipient.

As used herein the term “active principle” or “active ingredient” are used interchangeably. As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. Sterile injectable solutions are prepared by incorporating the agent of the present invention in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The present invention also relates to the use of a ET_{B} agonist of the present invention for the preparation of bioprosthetic valve. In this respect, the invention relates more particularly to bioprosthetic valve comprising an amount of a ET_{B} agonist. Such a local biomaterial or medical delivery device can be used to treat aortic valve stenosis. Such biomaterial or medical delivery device may be biodegradable. The agonist of the invention is preferably entrapped into the cusps of the valve. The cusps of the valve are generally made from tissue of mammals such as, without limitation, pigs (porcine), cows (bovine), horses, sheep, goats, monkeys, and humans. With said entrapment, it is possible to achieve a high level of local action. According to the present invention, the valve may be a collapsible elastical valve having one or more cusps and the collapsible elastical valve may be mounted on an elastical stent. In some embodiments, the collapsible elastical valve may comprise one or more cusps of biological origin. In some embodiments, the one or more cusps are porcine, bovine, or human. The elastical stent portion of the valve prosthesis used in the present invention may be self-expandable or expandable by way of a balloon catheter. The elastical stent may comprise any biocompatible material known to those of ordinary skill in the art. Examples of biocompatible materials include, but are not limited to, ceramics; polymers; stainless steel; titanium; nickel-titanium alloy, such as nitinol; tantalum; alloys containing cobalt, such as Elgiloy® and Phynox®; and the like. The process of disposing the coating composition which comprises the RAR agonist of the present invention may be any process known in the art. The local delivery according to the present invention allows for high concentration of the agonist of the present invention at the disease site with low concentration of circulating compound. For purposes of the invention, a therapeutically effective amount will be administered.

The invention will be further illustrated by the following figures and examples. However, these examples and figures should not be interpreted in any way as limiting the scope of the present invention.

### FIGURES

FIG. 1: Calcium content of valvular interstitial cells (VIC) cultured in procalcifying conditions containing inorganic phosphate (Pi) in absence and in presence of antagonists of either the ET_{A} or ET_{B} receptors and a agonist of the ET_{B} receptor. ET_{B} receptor antagonist increases the calcium content of VIC while the ET_{B} receptor agonist IRL 1620 decreases their calcium content compared to non-treated VIC.

### EXAMPLE

### Introduction

Calcified aortic stenosis (AS) is the most common acquired valvulopathy for which there is still no pharmacological treatment. Endothelin-1 (ET-1) is not only a powerful vasoconstrictor but also a pro-inflammatory and pro-fibrotic peptide whose role in AS remains unclear.

### Objective

The aim of this study was to characterize the role of ET-1 in the aortic valve calcification.

### Methods

Valvular endothelial cells (VEC), isolated from human aortic valves, were cultured in a cell perfusion system to assess ET-1 production in different fluid flow shear stress conditions. In addition, valvular interstitial cells (VIC) were cultured in a pro-calcifying culture medium containing inorganic phosphate (Pi) with or without antagonists of either the ET_{A} or ETB receptor antagonists or with the agonist of the ET_{B} receptor IRL 1620 during 10 days. Aortic valves from rats were also cultured in ex vivo and stimulated with ET-1 antagonists. Calcium content was assessed using an o-cresolphtalein-based assay and fluorescence by Osteosens. VEC prepro-ET-1 and VIC osteogenic mRNA expression levels were evaluated by RTqPCR.

### Results

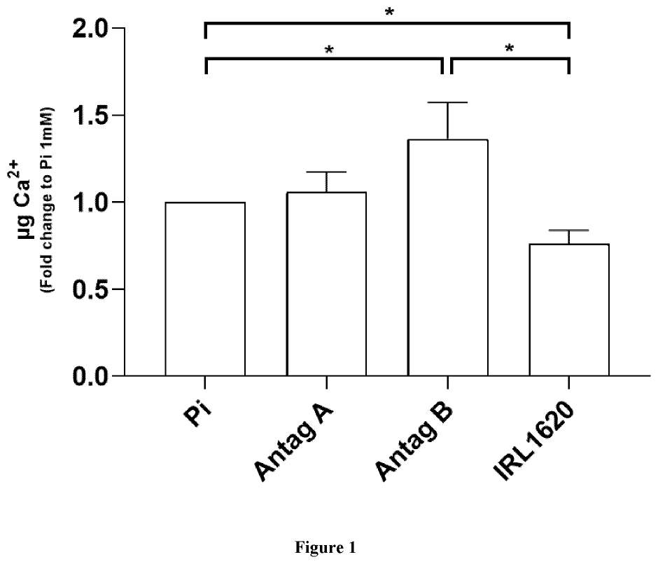

Turbulent shear stress, mimicking the flow conditions suffered by the valve at the aortic side increased VEC prepro-ET-1 mRNA expression level and ET-1 release compared to laminar shear stress. Calcium content and fluorescence by Osteosens staining of VIC and aortic valves were increased after blockade of ET_{B} receptor and this effect was potentiated by concomitant blockade of ET_{A} receptor. In contrast, the ET_{B} receptor agonist IRL-1620 decreased the calcium content of VIC (FIG. 1). The mRNA expression of osteopontin, RUNX2, and BMP2 was similarly increased by ET_{A} and ET_{B} blockade.

### Conclusion

These results support a protective role of the endothelin system against the development of AS. Further studies are warranted to characterize the intracellular pathways and to confirm these results in in vivo models.

### REFERENCES

Throughout this application, various references describe the state of the art to which this invention pertains. The disclosures of these references are hereby incorporated by reference into the present disclosure.

1. A method of treating Aortic Valve Stenosis (AS) in a patient in need thereof comprising administering to the patient a therapeutically effective amount of an endothelin receptor type B (ET_{B}) agonist.

2. A method of reversing, preventing, or delaying calcification of an aortic valve in a patient in need thereof comprising administering to the patient a therapeutically effective amount of an endothelin receptor type B (ET_{B}) agonist.

3. The method of claim 2 wherein the aortic valve is an implanted bioprosthetic valve.

4. The method of claim 3, wherein the method prevents degeneration of the implanted bioprosthetic valve.

5. The method of claim 3, wherein the method delays or prevents calcification of the implanted bioprosthetic valve after valve replacement either surgically or after transcatheter aortic valve implantation (TAVI).

6. The method of claim 1 wherein the patient suffers from a calcific aortic valve disease (CAVD).

7. The method according to claim 1 wherein the endothelin receptor type B agonist is chosen from sarafotoxin 6c, IRL-1620, Endothelin 3, BQ-3020 and Ala(1,3,11,15)-Endothelin-1.

8. The method according to claim 1 wherein the endothelin receptor type B agonist is IRL-1620.

9. A method of making a bioprosthetic valve, comprising preparing the biosynthetic valve and entrapping a therapeutically effective amount of an endothelin receptor type B agonist into cusps of the valve.

10. A bioprosthetic valve comprising a therapeutically effective amount of an endothelin receptor type B agonist.

11. The bioprosthetic valve of claim 10 wherein the endothelin receptor type B agonist is entrapped into cusps of the valve.

12. A method of preventing or treating aortic valve stenosis in a patient in need thereof, comprising implanting the bioprosthetic valve of claim 10 into the patient.

13. The method according to claim 2, wherein the patient suffers from a calcific aortic valve disease (CAVD).

14. The method according to claim 2, wherein the endothelin receptor type B agonist is chosen from sarafotoxin 6c, IRL-1620, Endothelin 3, BQ-3020 and Ala(1,3,11,15)-Endothelin-1.

15. The method according to claim 2, wherein the endothelin receptor type B agonist is IRL-1620.
